(19) United States
(12) Patent Application Publication
(10) Pub. No.: US 2023/0309883 A1
(43) Pub. Date: Oct. 5, 2023

(54) SYSTEM AND METHOD FOR CONDUCTING MENTAL HEALTH ASSESSMENT AND EVALUATION, MATCHING NEEDS, AND PREDICTING CONTENT AND EXPERIENCES FOR IMPROVING MENTAL HEALTH

(71) Applicant: BeMe Health, Inc., Marblehead, MA (US)

(72) Inventors: Mandeep DHILLON, Palmer, PR (US); Nicoletta TESSLER, Surfside, FL (US); Prabhanjan C. GURUMOHAN, Palo Alto, CA (US); Elisha FERGUSON, Brooklyn, NY (US); Danielle RAMO-LARIOS, Oakland, CA (US); Tali McKell BEESLEY, Pagosa Springs, CO (US); Vivien Keil ANDERSON, San Diego, CO (US); Neha Praveen CHAUDHARY, San Francisco, CA (US)

(73) Assignee: BeMe Health, Inc., Marblehead, MA (US)

(21) Appl. No.: 18/129,794

(22) Filed: Mar. 31, 2023

Related U.S. Application Data
(60) Provisional application No. 63/326,646, filed on Apr. 1, 2022.

(51) Int. Cl. A61B 5/16; G16H 20/70; G16H 50/20

(52) U.S. Cl. CPC A61B 5/165; G16H 20/70; G16H 50/20

(57) ABSTRACT

A system for conducting mental health assessment and/or evaluation, matching needs, and predicting content and experiences for improving mental health is provided. In particular, the system collects various signals relating to mood, mental health, and/or other characteristics associated with a user. The system labels the signals and/or interactions based on an assessment of how user engagement with specific content should be interpreted. The labeled interactions may be assigned a score value by the system to determine deficits or needs relating to the mental health of the user. Based on the score value and signals, the system determines content, activities, and/or other resources for the user to interact with that may assist in reducing the deficits and improving the user's mental health. The system uses artificial intelligence models to analyze the interactions and signals in real-time and adjusts the recommended content to enhance mental health over time.

### CROSS-REFERENCE TO RELATED APPLICATIONS

The present application claims priority to and the benefit of U.S. Provisional Patent Application No. 63/326,646, filed on Apr. 1, 2022, the entirety of which is hereby incorporated by reference.

### FIELD OF THE INVENTION

The present application relates to mental health enhancement technologies, data analytics technologies, artificial intelligence technologies, machine learning technologies, cloud-computing technologies, signal processing technologies, sensor technologies, interactive technologies, and, more particularly, to a system and method for conducting mental health evaluation and assessment, matching needs, and predicting content and experiences for improving mental health.

### BACKGROUND

Today, it has become increasingly important to be able to gather information associated with mental health, evaluate and assess mental health, and implement measures to improve mental health in a more effective and efficient manner. A variety of factors may affect an individual's mental health. For example, such factors may include, but are not limited to, physical health, physical and substance abuse, self-esteem, confidence, environmental factors, food intake, relationships, activity levels, genetic factors, and/or a variety of other factors. Typically, an individual may seek assistance with improving his or her mental health by contacting and arranging appointments with mental health professionals, such as therapists, psychiatrists, and psychologists. Such mental health professionals typically assess and/or evaluate the mental health of an individual during a mental health session at a clinical or office setting. Assessments relating to mental health may be made by the professionals based on questions posed to individuals, observations relating to responses provided by individuals, and analyzing the responses and observations based on their mental health expertise. Certain assessment tests, such as GAD7, PHQ9, PSS4 and other types of tests have been widely used by mental health professionals to screen for mental health conditions and track changes in symptom severity over time. In recent times, such professionals and technology companies have employed the use of software applications for content delivery and telemedicine to connect mental health patients to their care provider.

Nevertheless, despite the foregoing, there remains room for substantial enhancements to existing technologies and processes and for the development of new technologies and processes to enhance mental health and overall well-being. While currently existing technologies provide for various benefits, such technologies still come with various drawbacks and inefficiencies. For example, currently existing processes and technologies often do not detect or prevent mental health declines early enough or fast enough to have meaningful impact, especially when it comes to reducing potential negative consequences, such as lasting depression or suicide. Additionally, existing mental health processes often require a significant amount of administrative and clinical work to schedule appointments, schedule follow-up sessions, generate diagnoses, and/or generate mental health wellness plans tailored to each individual seeking mental health assistance. Additionally, there are often significant gaps in time between mental health sessions conducted with mental health professionals, and such gaps may lead to regressions in mental health. Furthermore, while currently existing processes may have short-term effectiveness on a case-by-case basis, existing technologies often fail to have a lasting effect on stabilizing or improving mental health. Moreover, existing technologies fail to take advantage of artificial intelligence technologies that could assist a system in adapting to changing mental health needs. Based on the foregoing, current technologies and processes may be enhanced in order to provide for more effective monitoring, greater quality data, faster detection of mental health changes, more effective mental health intervention processes, higher quality predictive capabilities, and more effective interactions with individuals. Such enhancements and improvements to processes and technologies may provide for enhanced mental health wellness compliance, increased individual satisfaction with mental wellness programs, and, ultimately, improved mental health for individuals.

### SUMMARY

A system and accompanying methods for conducting mental health assessment and/or evaluation, matching needs, and predicting content and experiences for improving mental health are disclosed. In particular, the system and methods utilize devices and applications in combination with artificial intelligence models to provide a unique and different ability to assess, evaluate, and improve the mental health status of individuals interacting with the system. In certain embodiments, the system and methods may provide such an ability to assess, evaluate, and improve mental health of individuals without the need for explicit questions or the presence of a physician or therapist with the individuals. In operation, the system and methods may include capturing signals, content and/or data associated with an individual's mood and/or mental state from devices, applications, and/or systems that are utilized to interact with individuals. Additionally, in certain embodiments, other engagement conducted by individuals with an application, such as an individual's choice of content, participation in activities, completion of activities or content, along with the captured signals, content and/or data may be utilized by the system and methods to assess and/or evaluate an individual's mental health and/or wellness. In certain embodiments, signals including any amount of the content and/or data may be labeled based on an assessment of how the individual's engagement with specific content should be interpreted based on a detailed framework determined using, for example, behavioral health experts.

The labeled signals (and/or interactions) may be assigned a score value that assists in determining a specific mental health deficit or need that an individual might have that may be addressed by providing additional content or interactions with the applications, devices, and/or systems. As the individual conducts additional interactions with additional content on the applications, devices, and/or systems, the system and methods may include dynamically recalculating the score value with each interaction, and the recalculated score value may be utilized to predict what further pieces of content, care activities, coaching, crisis support, therapy, and/or other potential mental health recommendations may be best suited to improve the individuals mental health state and overall well-being over time.

In certain embodiments, data and/or content associated with individuals and the individual's interactions with the system, applications, and/or devices may be loaded into artificial intelligence models that have been trained to recognize patterns, behaviors, moods, feelings, actions, and/or other detectable characteristics associated with mental health. Such artificial intelligence models may be trained to recognize the patterns, behaviors objects, activities, individuals, and/or other items of interest based on analyzing other content and/or data that have been fed into the models on previous occasions. The effectiveness and detection capability of the artificial intelligence models may be enhanced as the models receive additional content and/or data over time, such as content and/or data resulting from further interactions with the individual. The captured content and/or data may be compared to the content and/or data used to train the models and/or to deductions, reasoning, intelligence, correlations, outputs, analyses, and/or other information that the artificial intelligence model(s) learned based on the content and/or data used to train the models. The score values, mental health assessments and/or evaluations, and/or predictions may be generated using the artificial intelligence model(s) and machine learning. The labels, scores, assessments, evaluations, and/or mood improvement objective functions may be utilized to promote emotional and/or mental wellness.

Based on at least the foregoing capabilities, the system and methods may provide a dynamic system of measurement and prediction that uniquely provides for significantly greater understanding of an individual's mental wellness without the use of therapists or exclusive reliance on traditional mental health assessments. Such a system significantly broadens the number of signals utilized to assess, evaluate, and/or improve mental health, increases the speed of signal collection, dynamically uses big data to refine and calibrate mental health recommendations, and, ultimately, creates an implementable and personalized objective function that assists an individual in improving mental health and well-being through recommended content, care activities, coaching, community, crises resources, therapy referrals, and other resources.

In one embodiment, a system for conducting mental health assessment, matching needs, and predicting content and experiences for improving mental health is provided. The system may include a memory that stores instructions and a processor that executes the instructions to perform various operations of the system. The system may perform an operation that includes capturing, via an application, a plurality of signals associated with a mood, a mental state, or a combination thereof, associated with a user. Signals including information relating to a user's interaction with content may also be captured via the system. Additionally, the system may perform an operation that includes labeling the plurality of signals based on an assessment of how engagement with content is to be interpreted. The system may then perform an operation that includes assigning, based on the plurality of signals, a score value relating to a mental health of the user. The system may proceed to perform an operation that includes determining, based on the score value, the plurality of signals, or a combination thereof, a deficit or need of the user relating to the mental health of the user. The system may perform an operation that includes determining, by utilizing an artificial intelligence model, a prediction relating to content, activities, resources, or a combination thereof, to be presented or delivered for interaction with the user to enhance the score value relating to the mental health of the user. Furthermore, the system may perform an operation that includes providing access to the content, activities, resources, or a combination thereof, to a device of the user. Moreover, the system may perform an operation that includes dynamically adjusting the score value relating to the mental health of the user in real-time as the user interacts with the content, activities, resources, or a combination thereof.

In another embodiment, a method for conducting mental health assessment, matching needs, and predicting content and experiences for improving mental health is disclosed. The method may include a memory that stores instructions and a processor that executes the instructions to perform the functionality of the method. In particular, the method may include capturing, via an application, a plurality of signals associated with a mood, a mental state, or a combination thereof, associated with a user. Information relating to a user's interaction with content may also be captured via the method. Additionally, the method may include labeling the plurality of signals based on an assessment of how engagement with content is to be interpreted. The method may include assigning, based on the plurality of signals, a score value relating to a mental health of the user. The method may proceed to include determining, based on the score value, the plurality of signals, or a combination thereof, a deficit or need of the user relating to the mental health of the user. Also, the method may include determining, by utilizing an artificial intelligence model, a prediction relating to content, activities, resources, or a combination thereof, to be presented or delivered for interaction with the user to enhance the score value relating to the mental health of the user. Furthermore, the method may include providing access to the content, activities, resources, or a combination thereof, to a device of the user. Moreover, the method may include dynamically adjusting the score value relating to the mental health of the user in real-time as the user interacts with the content, activities, resources, or a combination thereof.

According to yet another embodiment, a computer-readable device comprising instructions, which, when loaded and executed by a processor cause the processor to perform operations, the operations comprising: capturing, via an application, a plurality of signals associated with a mood, a mental state, or a combination thereof, associated with a user, interaction data associated with interaction with content, or a combination thereof; labeling the plurality of signals based on an assessment of how engagement with content is to be interpreted; assigning, based on the plurality of signals, a score value relating to a mental health of the user; determining, based on the score value, the plurality of signals, or a combination thereof, a deficit or need of the user relating to the mental health of the user; determining, by utilizing an artificial intelligence model, a prediction relating to content, activities, resources, or a combination thereof, to be presented or delivered for interaction with the user to enhance the score value relating to the mental health of the user; providing access to the content, activities, resources, or a combination thereof, to a device of the user; and dynamically adjusting the score value relating to the mental health of the user in real-time as the user interacts with the content, activities, resources, or a combination thereof.

These and other features of the systems and methods for conducting mental health assessment, matching needs, and predicting content and experiences for improving mental health are described in the following detailed description, drawings, and appended claims.

### BRIEF DESCRIPTION OF THE DRAWINGS

FIG. 1 is a schematic diagram of a system for conducting mental health assessment, matching needs, and predicting content and experiences for improving mental health according to an embodiment of the present disclosure.

FIG. 2 is an exemplary illustration of various characteristics of and variables for use with an algorithm to facilitate the operative functionality of the system of FIG. 1.

FIG. 3 illustrates exemplary user interface screenshots of an application for use with the system of FIG. 1.

FIG. 4 illustrates exemplary user interface screenshots of an application that facilitates interaction with users of the system of FIG. 1.

FIG. 5 illustrates exemplary user interface screenshots illustrating the ability to obtain feedback from a user of the system of FIG. 1.

FIG. 6 illustrates exemplary user interface screenshots illustrating a sample activity for a user to participate in to improve mental health according to certain embodiments of the present disclosure.

FIG. 7 is a flow diagram illustrating a sample method for conducting mental health assessment, matching needs, and predicting content and experiences for improving mental health according to an embodiment of the present disclosure.

FIG. 8 is a schematic diagram of a machine in the form of a computer system within which a set of instructions, when executed, may cause the machine to for conducting mental health assessment, matching needs, and predicting content and experiences for improving mental health.

### DETAILED DESCRIPTION OF THE DRAWINGS

A system 100 and accompanying methods (e.g., method 700) for conducting mental health assessment, matching needs, and predicting content and experiences for improving mental health are disclosed. In particular, the system 100 and methods utilize devices and applications in combination with artificial intelligence models to provide a unique and different ability to assess, evaluate, and/or improve the mental health statuses of individuals interacting with the system 100. In certain embodiments, the system 100 and methods may provide such an ability to assess, evaluate, and/or improve mental health of individuals without the need for explicit questions or the presence of a physician or therapist with the individuals. In operation, the system and methods may include capturing signals, content and/or data associated with an individual's mood and/or mental state from devices, applications, and/or systems that are utilized to interact with individuals. Additionally, in certain embodiments, other engagement conducted by individuals with an application, such as an individual's choice of content, participation in activities, completion of activities or content, along with the captured signals, content and/or data may be utilized by the system 100 and methods to assess and/or evaluate an individual's mental health and/or wellness. In certain embodiments, signals including any amount of the content and/or data may be labeled based on an assessment of how the individual's engagement with specific content should be interpreted based on a detailed framework determined using, for example, behavioral health experts.

The system 100 and methods may assign a score value to the labeled signals (and/or interactions) that assists in determining a specific mental health deficit or need that an individual might have that may be addressed by providing additional content or interactions with the applications, devices, and/or systems. As the individual conducts additional interactions with additional content on the applications, devices, and/or systems, the system 100 and methods may include dynamically recalculating the score value with each interaction, and the recalculated score value may be utilized to predict what further pieces of content, care activities, coaching, crisis support, therapy, and/or other potential mental health recommendations may be best suited to improve the individuals mental health state and overall well-being over time.

In certain embodiments, data and/or content associated with individuals and the individual's interactions with the system 100, applications, and/or devices may be loaded into artificial intelligence models that have been trained to recognize patterns, behaviors, moods, feelings, actions, and/or other detectable characteristics associated with mental health. Such artificial intelligence models may be trained to recognize the patterns, behaviors objects, activities, individuals, and/or other items of interest based on analyzing other content and/or data that have been fed into the models on previous occasions. The effectiveness and detection capability of the artificial intelligence models may be enhanced as the models receive additional content and/or data over time, such as content and/or data resulting from further interactions with the individual or other individuals, such as individuals that may have a correlation with the mental health of the individual. The captured content and/or data may be compared to the content and/or data used to train the models and/or to deductions, reasoning, intelligence, correlations, outputs, analyses, and/or other information that the artificial intelligence model(s) learned based on the content and/or data used to train the models. The score values, mental health assessments and/or evaluations, and/or predictions may be generated using the artificial intelligence model(s) and machine learning. The labels, scores, assessments, evaluations, and/or mood improvement objective functions may be utilized to promote emotional and/or mental wellness.

As a result, the system 100 and methods may provide a dynamic system of measurement and prediction that uniquely provides for significantly greater understanding of an individual's mental wellness without the use of therapists or exclusive reliance on traditional mental health assessments. Such a system 100 significantly broadens the number of signals utilized to assess, evaluate, and/or improve mental health, increases the speed of signal collection, dynamically uses big data to refine and calibrate mental health recommendations, and, ultimately, creates an implementable and personalized objective function that assists an individual in improving mental health and well-being through recommended content, care activities, coaching, community, crises resources, therapy referrals, and other resources.

In certain embodiments, a system for conducting mental health assessment, matching needs, and predicting content and experiences for improving mental health is provided. In certain embodiments, the system may include a memory that stores instructions; and a processor that executes the instructions to perform operations. In certain embodiments, the operations may include capturing, via an application, a plurality of signals associated with a mood, a mental state, or a combination thereof, associated with a user, interaction data associated with the user, or a combination thereof. In certain embodiments, the operations may include labeling the plurality of signals based on an assessment of how engagement with content is to be interpreted. In certain embodiments, the operations may include assigning, based on the plurality of signals, a score value relating to a mental health of the user. In certain embodiments, the operations may include determining, based on the score value, the plurality of signals, or a combination thereof, a deficit or need of the user relating to the mental health of the user. In certain embodiments, the operations may include determining, by utilizing an artificial intelligence model, a prediction relating to content, activities, resources, or a combination thereof, to be presented or delivered for interaction with the user to enhance the score value relating to the mental health of the user. In certain embodiments, the operations may include providing access to the content, activities, resources, or a combination thereof, to a device of the user. In certain embodiments, the operations may include dynamically adjusting the score value relating to the mental health of the user in real-time as the user interacts with the content, activities, resources, or a combination thereof.

In certain embodiments, the operations may include dynamically adjusting recommendations for content, activities, resources, or a combination thereof, in real-time as the score value is adjusted, as the user interacts with the content, activities, or resources, as circumstances relating to the user change, or a combination thereof. In certain embodiments, the content, activities, or resources may include one or more of a coaching session, a crisis resource, a community resource, a care activity, a therapy session, video content, gaming content, audio content, virtual reality content, augmented reality content, medical content, medical therapy sessions, surveys, mental health content, training content, or a combination thereof. In certain embodiments, the score value may include score values relating to a plurality of variables utilized in determining the mental health of the user. In certain embodiments, the plurality of variables may include one or more of a relationship variable, a physical variable (e.g., variables relating to physical aspects and/or characteristics of the user), a mind variable (e.g., variables relating to the mind of the user), a positivity variable (e.g., variables relating to the positivity of a user), an engagement variable (e.g., variables relating to whether a user engages with recommended content and/or activities), a mood tracker variable (e.g., variables relating to the mood of the user being tracked), a mental state variable (e.g., variables indicative of the mental state of the user), or a combination thereof.

In certain embodiments, the operations may include capturing the plurality of signals from the device of the user as the user interacts with the application. In certain embodiments, the operations may include generating the content, activities, resources, or a combination thereof, such that the content, resources, or a combination thereof, are tailored to improving the mental health of the user. In certain embodiments, the operations may include capturing the plurality of signals on a continuous basis or while the user is utilizing the application. In certain embodiments, the operations may include training the artificial intelligence model based on information associated with the user and/or other users, information known to be associated with mental health, patterns associated with generating mental health diagnoses, information associated types of content to enhance mental health, or a combination thereof. In certain embodiments, the operations may include adjusting a confidence score for the prediction based on whether the content, the activities, or the resources, affected the mental health of the user in a manner as predicted.

In certain embodiments, the operations may include visually rendering the score value in a graph on a user interface of the device of the user. In certain embodiments, the operations may further include visually rendering sub-scores of variables (e.g., scores for each of the variables that are utilized to calculate the score value) utilized in determining the score in the graph. In certain embodiments, the operations may include determining which content, activities, or resources generated the highest improvement to the score value relating to the mental health of the user. In certain embodiments, the operations may include providing a higher weight to the content, the activities, or the resources that generated the highest improvement to the score value in comparison to other content, other activities, or other resources.

In certain embodiments, a method for conducting mental health assessment, matching needs, and predicting content and experiences for improving mental health is provided. In certain embodiments, the method may include capturing, via an application and by utilizing instructions from a memory that are executed by a processor, a plurality of signals associated with a mood, a mental state, or a combination thereof, associated with a user, interaction data associated with the user, or a combination thereof. In certain embodiments, the method may include labeling the plurality of signals based on an assessment of how engagement with content is to be interpreted. In certain embodiments, the method may include assigning, based on the plurality of signals, a score value relating to a mental health of the user. In certain embodiments, the method may include determining, based on the score value, the plurality of signals, or a combination thereof, a deficit or need of the user relating to the mental health of the user. In certain embodiments, the method may include determining, by utilizing an artificial intelligence model, a prediction relating to content, activities, resources, or a combination thereof, to be presented or delivered for interaction with the user to enhance the score value relating to the mental health of the user. In certain embodiments, the method may include providing access to the content, activities, resources, or a combination thereof, to a device of the user. In certain embodiments, the method may include dynamically adjusting the score value relating to the mental health of the user in real-time as the user interacts with the content, activities, resources, or a combination thereof.

In certain embodiments, the method may include training the artificial intelligence model based on interactions with the content, the activities, the resources, or a combination thereof. In certain embodiments, the method may include providing access to the content, activities, resources, or a combination thereof, to the device of the user via a user interface of the application. In certain embodiments, the method may include recommending content, activities, resources, or a combination thereof, to the user based on the user having a threshold correlation with characteristics of another user. In certain embodiments, the method may include updating the adjusting a predictive capability of the artificial intelligence model based on interactions conducted by the user with respect to the content, activities, resources, or a combination thereof. In certain embodiments, the method may include capturing sensor data including biometric data, visual content of the user, audio content associated with the user, virtual reality content associated with the user, augmented reality content associated with the user, or a combination thereof, and utilize the sensor data to determine the deficit or need of the user relating to the mental health of the user. In certain embodiments, the method may include visually rendering adjustments to the score value to a user interface of the device of the user in real-time to advise the user to the adjustments to the score value based on interactions with the content, the activities, the resources, or a combination thereof.

A computer-readable device comprising instructions, which, when loaded and executed by a processor, cause the processor to perform operations, the operations comprising: capturing, via an application, a plurality of signals associated with a mood, a mental state, or a combination thereof, associated with a user, interaction data associated with the user, or a combination thereof; labeling the plurality of signals based on an assessment of how engagement with content is to be interpreted; assigning, based on the plurality of signals, a score value relating to a mental health of the user; determining, based on the score value, the plurality of signals, or a combination thereof, a deficit or need of the user relating to the mental health of the user; determining, by utilizing an artificial intelligence model, a prediction relating to content, activities, resources, or a combination thereof, to be presented or delivered for interaction with the user to enhance the score value relating to the mental health of the user; providing access to the content, activities, resources, or a combination thereof, to a device of the user; and dynamically adjusting the score value relating to the mental health of the user in real-time as the user interacts with the content, activities, resources, or a combination thereof.

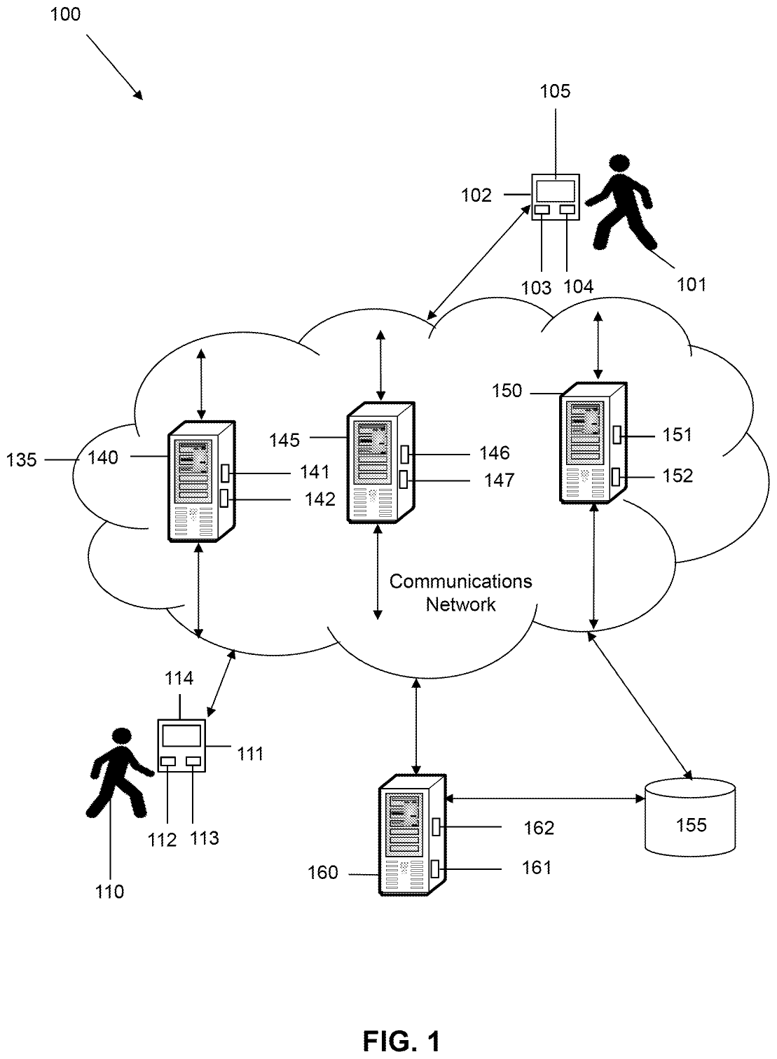

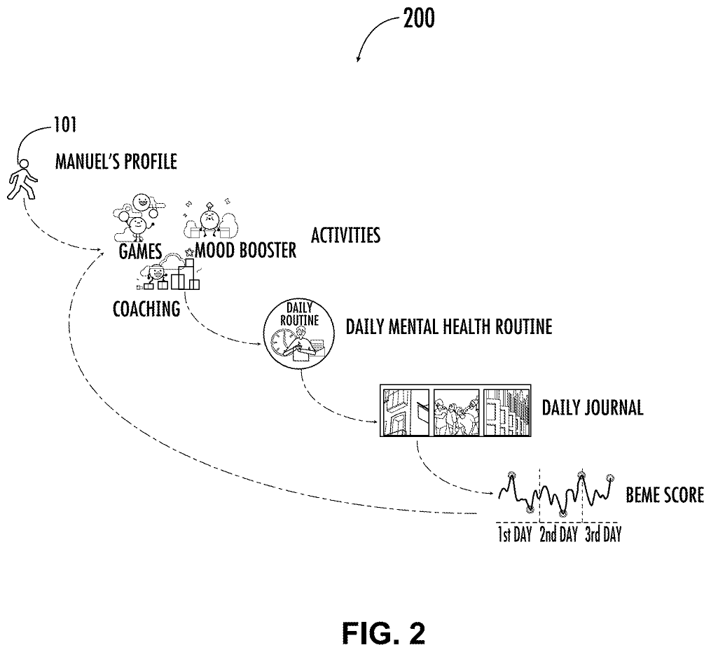

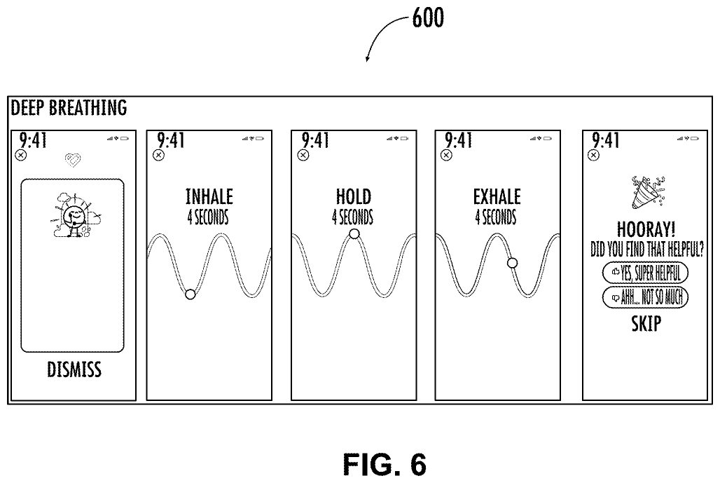

As shown in FIG. 1 and referring also to FIGS. 2-8, a system 100 for conducting mental health assessment, matching needs, and predicting content and experiences for improving mental health are disclosed. Notably, the system 100 may be configured to support, but is not limited to supporting, mental health systems and services, mental health improvement systems and services, monitoring systems and services, alert systems and services, data analytics systems and services, data collation and processing systems and services, artificial intelligence services and systems, machine learning services and systems, security systems and services, content delivery services, cloud computing services, satellite services, telephone services, voice-over-internet protocol services (VoIP), software as a service (SaaS) applications, platform as a service (PaaS) applications, gaming applications and services, social media applications and services, operations management applications and services, productivity applications and services, mobile applications and services, and/or any other computing applications and services. Notably, the system 100 may include a first user 101, who may utilize a first user device 102 to access data, content, and services, or to perform a variety of other tasks and functions. As an example, the first user 101 may utilize first user device 102 to transmit signals to access various online services and content, such as those available on an internet, on mobile devices, on other devices, and/or on various computing systems. As another example, the first user device 102 may be utilized to access an application, devices, and/or components of the system 100 that provide any or all of the operative functions of the system 100. In certain embodiments, the first user 101 may be any type of person, a robot, a humanoid, a program, a computer, any type of user, or a combination thereof. In certain embodiments, the first user 101 may be a person that may want to have their mental health assessed and/or evaluated, seek assistance with improving their mental health, and/or seek to participate in activities associated with fostering his or her mental health. The first user device 102 may include a memory 103 that includes instructions, and a processor 104 that executes the instructions from the memory 103 to perform the various operations that are performed by the first user device 102. In certain embodiments, the processor 104 may be hardware, software, or a combination thereof. The first user device 102 may also include an interface 105 (e.g. screen, monitor, graphical user interface, etc.) that may enable the first user 101 to interact with various applications executing on the first user device 102 and to interact with the system 100. In certain embodiments, the first user device 102 may be and/or may include a computer, any type of sensor, a laptop, a set-top-box, a tablet device, a phablet, a server, a mobile device, a smartphone, a smart watch, and/or any other type of computing device. Illustratively, the first user device 102 is shown as a smartphone device in FIG. 1. In certain embodiments, the first user device 102 may be utilized by the first user 101 to control and/or provide some or all of the operative functionality of the system 100.

In addition to using first user device 102, the first user 101 may also utilize and/or have access to additional user devices. As with first user device 102, the first user 101 may utilize the additional user devices to transmit signals to access various online services and content. The additional user devices may include memories that include instructions, and processors that executes the instructions from the memories to perform the various operations that are performed by the additional user devices. In certain embodiments, the processors of the additional user devices may be hardware, software, or a combination thereof. The additional user devices may also include interfaces that may enable the first user 101 to interact with various applications executing on the additional user devices and to interact with the system 100. In certain embodiments, the first user device 102 and/or the additional user devices may be and/or may include a computer, any type of sensor, a laptop, a set-top-box, a tablet device, a phablet, a server, a mobile device, a smartphone, a smart watch, and/or any other type of computing device, and/or any combination thereof. Sensors may include, but are not limited to, cameras, motion sensors, facial-recognition sensors, acoustic/audio sensors, pressure sensors, temperature sensors, light sensors, heart-rate sensors, blood pressure sensors, sweat detection sensors, breath-detection sensors, stress-detection sensors, any type of health sensor, humidity sensors, any type of sensors, or a combination thereof.

The first user device 102 and/or additional user devices may belong to and/or form a communications network. In certain embodiments, the communications network may be a local, mesh, or other network that enables and/or facilitates various aspects of the functionality of the system 100. In certain embodiments, the communications network may be formed between the first user device 102 and additional user devices through the use of any type of wireless or other protocol and/or technology. For example, user devices may communicate with one another in the communications network by utilizing any protocol and/or wireless technology, satellite, fiber, or any combination thereof. Notably, the communications network may be configured to communicatively link with and/or communicate with any other network of the system 100 and/or outside the system 100.

In certain embodiments, the first user device 102 and additional user devices belonging to the communications network may share and exchange data with each other via the communications network. For example, the user devices may share information relating to the various components of the user devices, information associated with images and/or content accessed by a user of the user devices, information identifying the locations of the user devices, information indicating the types of sensors that are contained in and/or on the user devices, information identifying the applications being utilized on the user devices, information identifying how the user devices are being utilized by a user, information identifying user profiles for users of the user devices, information identifying device profiles for the user devices, information identifying the number of devices in the communications network, information identifying devices being added to or removed from the communications network, any other information, or any combination thereof.

In addition to the first user 101, the system 100 may also include a second user 110. In certain embodiments, for example, the second user 110 may be another person that may seek to assess and/or evaluate her mental health and potentially improve upon her mental health and/or overall well-being. In certain embodiments, the second user 110 may be a mental health professional, such as, but not limited to, a psychiatrist, a therapist, a psychologist, and/or other mental health professional. In certain embodiments, the second user device 111 may be utilized by the second user 110 to transmit signals to request various types of content, services, and data provided by and/or accessible by communications network 135 or any other network in the system 100. In further embodiments, the second user 110 may be a robot, a computer, a vehicle, a humanoid, an animal, any type of user, or any combination thereof. The second user device 111 may include a memory 112 that includes instructions, and a processor 113 that executes the instructions from the memory 112 to perform the various operations that are performed by the second user device 111. In certain embodiments, the processor 113 may be hardware, software, or a combination thereof. The second user device 111 may also include an interface 114 (e.g. screen, monitor, graphical user interface, etc.) that may enable the first user 101 to interact with various applications executing on the second user device 111 and, in certain embodiments, to interact with the system 100. In certain embodiments, the second user device 111 may be a computer, a laptop, a set-top-box, a tablet device, a phablet, a server, a mobile device, a smartphone, a smart watch, and/or any other type of computing device. Illustratively, the second user device 111 is shown as a mobile device in FIG. 1. In certain embodiments, the second user device 111 may also include sensors, such as, but are not limited to, cameras, audio sensors, motion sensors, pressure sensors, temperature sensors, light sensors, heart-rate sensors, blood pressure sensors, sweat detection sensors, breath-detection sensors, stress-detection sensors, any type of health sensor, humidity sensors, any type of sensors, or a combination thereof.

In certain embodiments, the first user device 102, the additional user devices, and/or potentially the second user device 111 may have any number of software applications and/or application services stored and/or accessible thereon. For example, the first user device 102, the additional user devices, and/or potentially the second user device 111 may include applications for controlling and/or accessing the operative features and functionality of the system 100, applications for controlling and/or accessing any device of the system 100, interactive social media applications, biometric applications, cloud-based applications, VoIP applications, other types of phone-based applications, product-ordering applications, business applications, e-commerce applications, media streaming applications, content-based applications, media-editing applications, database applications, gaming applications, internet-based applications, browser applications, mobile applications, service-based applications, productivity applications, video applications, music applications, social media applications, any other type of applications, any types of application services, or a combination thereof. In certain embodiments, the software applications may support the functionality provided by the system 100 and methods described in the present disclosure. In certain embodiments, the software applications and services may include one or more graphical user interfaces so as to enable the first and/or potentially second users 101, 110 to readily interact with the software applications. The software applications and services may also be utilized by the first and/or potentially second users 101, 110 to interact with any device in the system 100, any network in the system 100, or any combination thereof. In certain embodiments, the first user device 102, the additional user devices, and/or potentially the second user device 111 may include associated telephone numbers, device identities, or any other identifiers to uniquely identify the first user device 102, the additional user devices, and/or the second user device 111.

The system 100 may also include a communications network 135. The communications network 135 may be under the control of a service provider, a business providing access to one or more applications supporting the functionality of the system 100, the first user 101, any other designated user, a computer, another network, or a combination thereof. The communications network 135 of the system 100 may be configured to link each of the devices in the system 100 to one another. For example, the communications network 135 may be utilized by the first user device 102 to connect with other devices within or outside communications network 135. Additionally, the communications network 135 may be configured to transmit, generate, and receive any information and data traversing the system 100. In certain embodiments, the communications network 135 may include any number of servers, databases, or other componentry. The communications network 135 may also include and be connected to a mesh network, a local network, a cloud-computing network, an IMS network, a VoIP network, a security network, a VoLTE network, a wireless network, an Ethernet network, a satellite network, a broadband network, a cellular network, a private network, a cable network, the Internet, an internet protocol network, MPLS network, a content distribution network, any network, or any combination thereof. Illustratively, servers 140, 145, and 150 are shown as being included within communications network 135. In certain embodiments, the communications network 135 may be part of a single autonomous system that is located in a particular geographic region, or be part of multiple autonomous systems that span several geographic regions.

Notably, the functionality of the system 100 may be supported and executed by using any combination of the servers 140, 145, 150, and 160. The servers 140, 145, and 150 may reside in communications network 135, however, in certain embodiments, the servers 140, 145, 150 may reside outside communications network 135. The servers 140, 145, and 150 may provide and serve as a server service that performs the various operations and functions provided by the system 100. In certain embodiments, the server 140 may include a memory 141 that includes instructions, and a processor 142 that executes the instructions from the memory 141 to perform various operations that are performed by the server 140. The processor 142 may be hardware, software, or a combination thereof. Similarly, the server 145 may include a memory 146 that includes instructions, and a processor 147 that executes the instructions from the memory 146 to perform the various operations that are performed by the server 145. Furthermore, the server 150 may include a memory 151 that includes instructions, and a processor 152 that executes the instructions from the memory 151 to perform the various operations that are performed by the server 150. In certain embodiments, the servers 140, 145, 150, and 160 may be network servers, routers, gateways, switches, media distribution hubs, signal transfer points, service control points, service switching points, firewalls, routers, edge devices, nodes, computers, mobile devices, or any other suitable computing device, or any combination thereof. In certain embodiments, the servers 140, 145, 150 may be communicatively linked to the communications network 135, any network, any device in the system 100, or any combination thereof.

The database 155 of the system 100 may be utilized to store and relay information that traverses the system 100, cache content that traverses the system 100, store data about each of the devices in the system 100 and perform any other typical functions of a database. In certain embodiments, the database 155 may be connected to or reside within the communications network 135, any other network, or a combination thereof. In certain embodiments, the database 155 may serve as a central repository for any information associated with any of the devices and information associated with the system 100. Furthermore, the database 155 may include a processor and memory or may be connected to a processor and memory to perform the various operation associated with the database 155. In certain embodiments, the database 155 may be connected to the servers 140, 145, 150, 160, the first user device 102, the second user device 111, the additional user devices, any devices in the system 100, any process of the system 100, any program of the system 100, any other device, any network, or any combination thereof.

The database 155 may also store information and metadata obtained from the system 100, store metadata and other information associated with the first and second users 101, 110, store artificial intelligence models utilized in the system 100, store sensor data and/or content obtained from an environment associated with the first and/or second users 101, 110, store predictions made by the system 100 and/or artificial intelligence models (e.g., predictions relating to which interactions are ideal for particular user, predictions relating to adjustments in scores for mental health based on types of interactions and/or activities that may be performed by a user, predictions relating to types of content that may be presented and/or delivered to a user to enhance mental health and/or scores, and/or any other predictions), store confidence scores relating to predictions made, store threshold values for confidence scores, store responses outputted and/or facilitated by the system 100, store information associated with anything detected, assessed, evaluated, and/or recommended via the system 100, store information and/or content utilized to train the artificial intelligence models, store information associated with behaviors and/or actions conducted by individuals with respect to the system 100, store user profiles associated with the first and second users 101, 110, store device profiles associated with any device in the system 100, store communications traversing the system 100, store user preferences, store information associated with any device or signal in the system 100, store information relating to patterns of usage relating to the user devices 102, 111, store any information obtained from any of the networks in the system 100, store historical data associated with the first and second users 101, 110, store device characteristics, store information relating to any devices associated with the first and second users 101, 110, store information associated with the communications network 135, store any information generated and/or processed by the system 100, store any of the information disclosed for any of the operations and functions disclosed for the system 100 herewith, store any information traversing the system 100, or any combination thereof. Furthermore, the database 155 may be configured to process queries sent to it by any device in the system 100.

Operatively, the system 100 may operate and/or execute the functionality as described in the methods (e.g. method 700 as described below) of the present disclosure. Additionally, the system 100 may incorporate the use of artificial intelligence models, machine learning, and/or neural networks. In some embodiments, the exemplary inventive computer-based systems/platforms, the exemplary inventive computer-based devices, and/or the exemplary inventive computer-based components of the present disclosure may be configured to utilize one or more exemplary AI/machine learning techniques chosen from, but not limited to, decision trees, boosting, support-vector machines, neural networks, nearest neighbor algorithms, Naive Bayes, bagging, random forests, and the like. In some embodiments and, optionally, in combination of any embodiment described above or below, an exemplary neutral network technique may be one of, without limitation, feedforward neural network, radial basis function network, recurrent neural network, convolutional network (e.g., U-net) or other suitable network. In some embodiments and, optionally, in combination of any embodiment described above or below, an exemplary implementation of Neural Network may be executed as follows: i) Define Neural Network architecture/model, ii) Transfer the input data to the exemplary neural network model, iii) Train the exemplary model incrementally, iv) determine the accuracy for a specific number of timesteps, v) apply the exemplary trained model to process the newly-received input data, vi) optionally and in parallel, continue to train the exemplary trained model with a predetermined periodicity.

In some embodiments and, optionally, in combination of any embodiment described above or below, the exemplary trained neural network model may specify a neural network by at least a neural network topology, a series of activation functions, and connection weights. For example, the topology of a neural network may include a configuration of nodes of the neural network and connections between such nodes. In some embodiments and, optionally, in combination of any embodiment described above or below, the exemplary trained neural network model may also be specified to include other parameters, including but not limited to, bias values/functions and/or aggregation functions. For example, an activation function of a node may be a step function, sine function, continuous or piecewise linear function, sigmoid function, hyperbolic tangent function, or other type of mathematical function that represents a threshold at which the node is activated. In some embodiments and, optionally, in combination of any embodiment described above or below, the exemplary aggregation function may be a mathematical function that combines (e.g., sum, product, etc.) input signals to the node. In some embodiments and, optionally, in combination of any embodiment described above or below, an output of the exemplary aggregation function may be used as input to the exemplary activation function. In some embodiments and, optionally, in combination of any embodiment described above or below, the bias may be a constant value or function that may be used by the aggregation function and/or the activation function to make the node more or less likely to be activated.

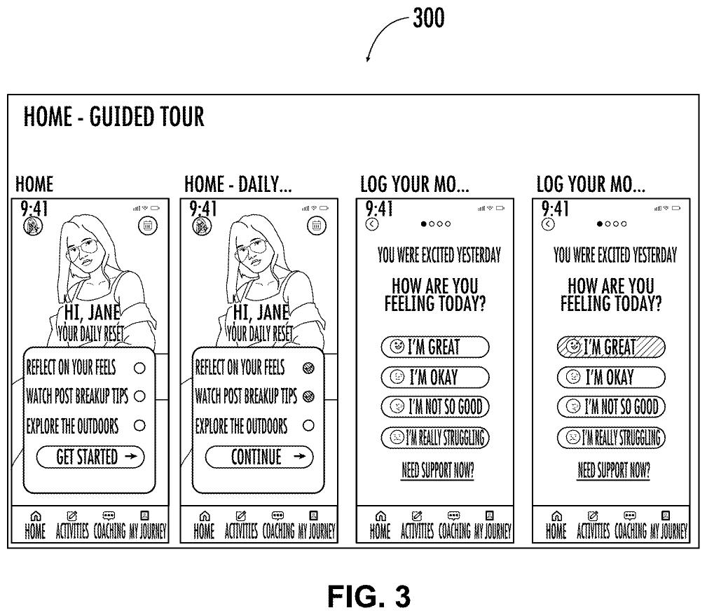

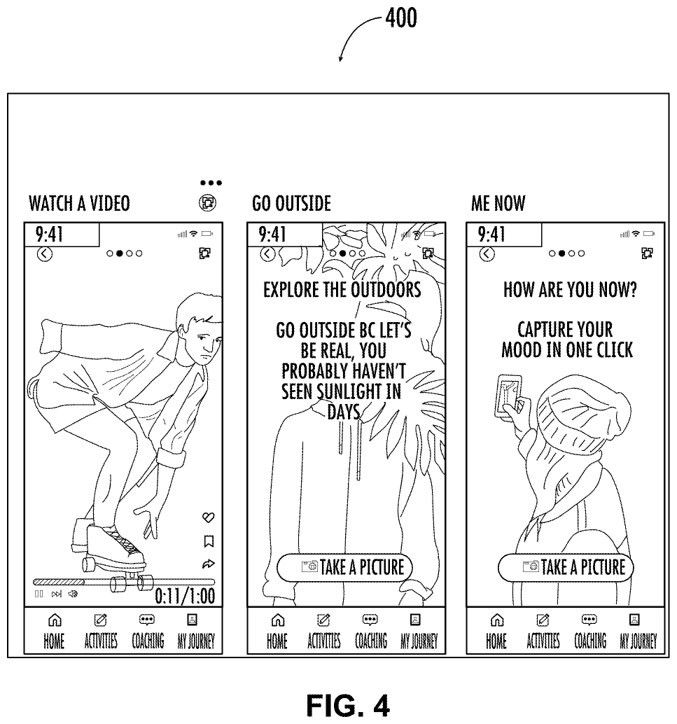

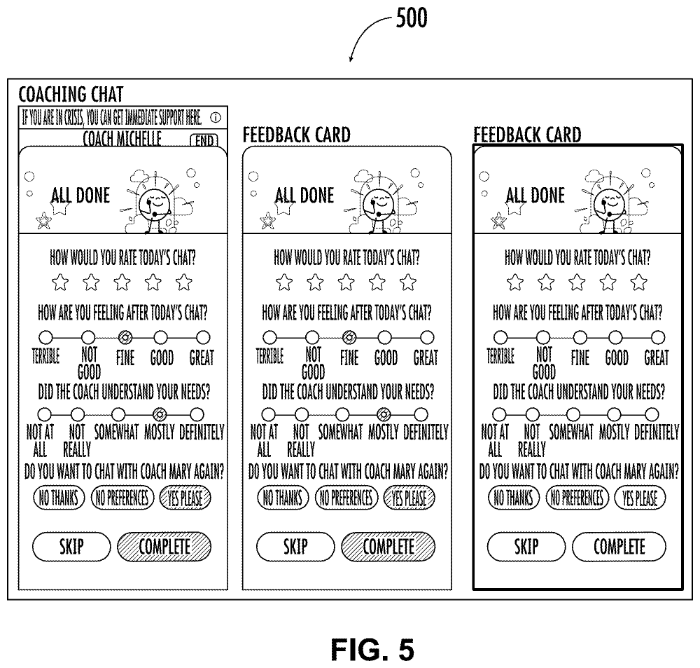

Referring now also to FIG. 2, an exemplary algorithmic process flow 200 for use with the system 100 for analyzing a user's profile, examining activities conducted by a user with an application of the system 100, determining a mental health routine for the user, compiling a daily journal of activities and/or interactions performed by the user, and then calculating the mental health score of the user is shown. User interface screen 300 of FIG. 3 illustrates sample user interfaces for getting a user started with a wellness program provided by the system 100 and obtaining inputs from the user relating to the user's mental health. User interface screen 400 of FIG. 4 illustrates sample content, activities, and/or other resources recommended by the system 100 to enhance a user's mental health and well-being. User interface screen 500 of FIG. 5 illustrates sample feedback forms for users to submit feedback relating to content, activities, and/or resources that the user may have interacted with to improve the user's mental health and well-being. The feedback may be utilized to track the effectiveness of such content, activities, and/or resources in achieving the expected change in mental health and/or well-being. User interface screen 600 illustrates a sample activities relating to breathing exercises provided by the application of the system 100 to enhance the user's mental health and well-being.

Notably, as shown in FIG. 1, the system 100 may perform any of the operative functions disclosed herein by utilizing the processing capabilities of server 160, the storage capacity of the database 155, or any other component of the system 100 to perform the operative functions disclosed herein. The server 160 may include one or more processors 162 that may be configured to process any of the various functions of the system 100. The processors 162 may be software, hardware, or a combination of hardware and software. Additionally, the server 160 may also include a memory 161, which stores instructions that the processors 162 may execute to perform various operations of the system 100. For example, the server 160 may assist in processing loads handled by the various devices in the system 100, such as, but not limited to, capturing signals from users and/or devices relating to mood, mental state, and/or other characteristics associated with the user; labeling the signals based on assessments of how engagement with specific content should be interpreted by the system 100; assigning score values to labeled interactions and/or categories associated with mental health to determine specific deficits or needs that an individual might have that may be addressed with further interactions recommended by the system 100; dynamically recalculating the score values in real-time as interactions are conducted by the user with applications of the system 100; utilizing artificial intelligence models to analyze data associated with mental health and/or interactions utilized to foster mental health to promote emotional wellness; calibrating and/or adjusting recommendations to enhance mental health in real-time; and performing any other suitable operations conducted in the system 100 or otherwise. In one embodiment, multiple servers 160 may be utilized to process the functions of the system 100. The server 160 and other devices in the system 100, may utilize the database 155 for storing data about the devices in the system 100 or any other information that is associated with the system 100. In one embodiment, multiple databases 155 may be utilized to store data in the system 100.

Although FIGS. 1-8 illustrates specific example configurations of the various components of the system 100, the system 100 may include any configuration of the components, which may include using a greater or lesser number of the components. For example, the system 100 is illustratively shown as including a first user device 102, a second user device 111, a communications network 135, a server 140, a server 145, a server 150, a server 160, and a database 155. However, the system 100 may include multiple first user devices 102, multiple second user devices 111, multiple communications networks 135, multiple servers 140, multiple servers 145, multiple servers 150, multiple servers 160, multiple databases 155, or any number of any of the other components inside or outside the system 100. Furthermore, in certain embodiments, substantial portions of the functionality and operations of the system 100 may be performed by other networks and systems that may be connected to system 100.

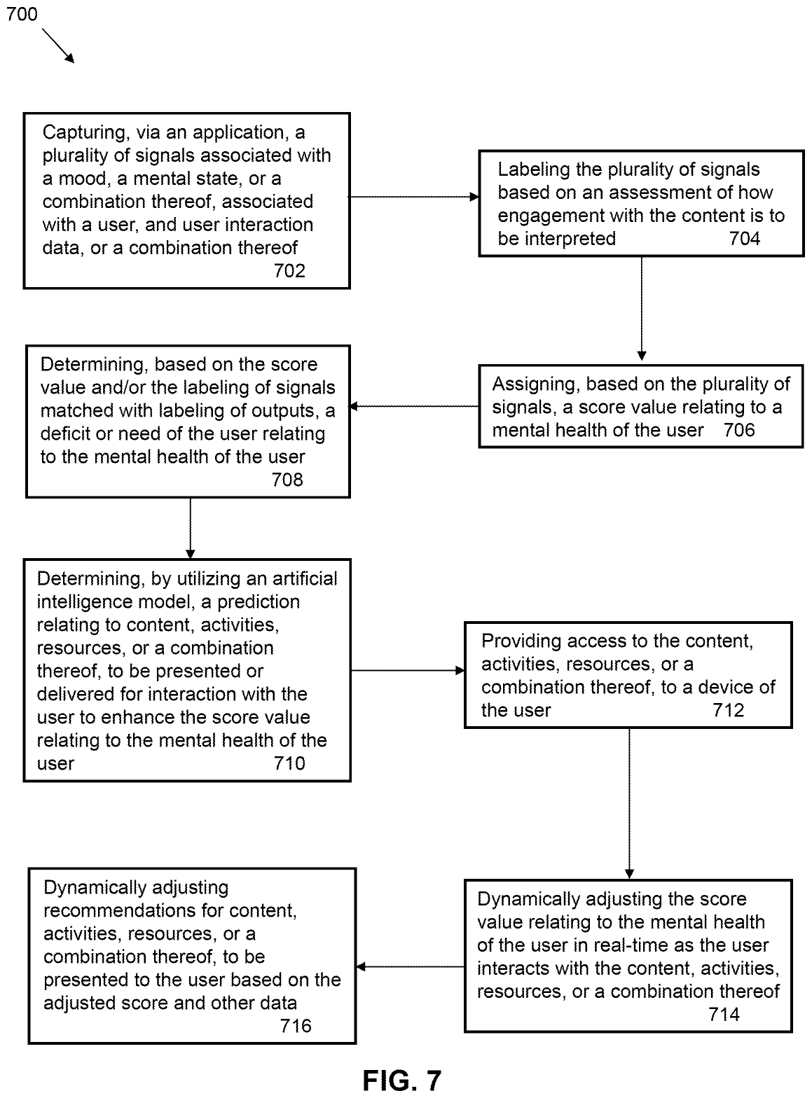

Notably, the system 100 may execute and/or conduct the functionality as described in the method(s) that follow. As shown in FIG. 7, an exemplary method 700 for conducting mental health assessment and/or evaluation, matching needs, and predicting content and experiences for improving mental heal is schematically illustrated. The method 700 and/or functionality and features supporting the method 700 may be conducted via an application of the system 100, devices of the system 100, processes of the system 100, any component of the system 100, or a combination thereof. The method 700 may include steps for analyzing data associated with users, determining mental health information for users, determining content to present or deliver to the users to enhance their mental health, and adjusting the content in real-time based on how the users' mental health changes over time. At step 702, the method 700 may include capturing, via an application, a plurality of signals associated with a mood, a mental state, or a combination thereof, associated with a user. In certain embodiments, the signals may include, but are not limited to, signals including information associated with any interaction by the user with the application, sensor data associated with the user obtained from sensors, information related to the user provided by a third party, device, and/or system, any other signals, or a combination thereof. In certain embodiments, for example, an interaction with the application may include the user responding to questions posed by the application to the user (e.g., how are you feeling today?), a device capturing media content associated with the user (e.g., the user and/or application takes an image of the user using the first user device 102 at a certain point in time that shows the user's expressions, the user and/or application records audio content of the user (e.g., speech), and the like), the user selecting an emoticon, image, or other media content (e.g., a blurble) expressing and/or indicating an emotion and/or mood that corresponds to the user's mood and/or emotion, the user providing responses to polls (e.g., micropolls that poll the user with questions seeking information relating to mood and/or emotion of the user, such as, but not limited to, demographic information, user feedback, and/or other information), the user providing responses to assessments provided by the application (e.g., assessments and/or quizzes that pose questions or request information from the user that may be utilized to determine the user's mood and/or emotional state), the user participating in mood boosting activities provided by the application (e.g., a mood boosting activity may prompt the user to exercise, listen to music and/or create a feel-good playlist, look at, interact with, and/or experience mood-boosting images and/or other content, participate in a coaching, chat, and/or therapy session with a professional, the user reviewing and/or reading blog content provided by the application, the user interacting with mood-tracking functionality of the application (e.g., modules and/or functionality of the application that periodically questions or requests information relating to the mood and/or emotion of the user), the user providing information that may be utilized to record a timeline feed (e.g., a personal journey) for the user relating to the activities and/or interactions that the user participates in, the user performing any type of interaction with the application and/or with other devices, systems, and/or individuals, or a combination thereof. In certain embodiments, the signals may be digital signals that include information associated with the interactions that may be utilized by the system 100 to determine a deficit or need of the user relating to the mental health of the user and to predict content, activities, and/or resources to be presented or delivered to the user to enhance the user's mood and/or emotional state.

In certain embodiments, at step 702 the method 700 may also capture information relating to how long and/or in what manner a user interacted with or perceived content served to them by the system 100. Such information may also include the interaction data gathered from users at step 714 described below. In certain embodiments, the capturing of signals may be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device.

At step 704, the method 700 may include labeling the plurality of signals based on an assessment of how engagement with the content is to be interpreted. In certain embodiments, for example, an assessment of how engagement with the content is to be interpreted may indicate that a user did not interact with content (or the application) as expected (e.g., instead of boosting the user's mood, the content actually did not improve the mood or made the mood worse), that the user did interact with content (or the application) as expected (e.g., the interaction was expected to be associated with happiness and the user was happy), that a certain type of interaction with content and/or the application is associated with a certain type of mood and/or emotion, or a combination thereof. In embodiments, labeling the signals may include categorizing each signal based on how it relates to a user's mental health. In certain embodiments, labeling a signal may include, but is not limited to, digitally labeling the signals as indicative of a particular mood, emotion, mental state, emotional state, or a combination thereof, for the user and/or users in general. In certain embodiments, the determination of what label to associate with a signal may be conducted by a machine learning model (or artificial intelligence model) that may determine that the signal contains information that matches, has a pattern in common with, and/or has characteristics in common with training information utilized to train the machine learning model that is known (or tagged or labeled) to be associated with a particular mood, emotion, mental state, and/or emotional state. In certain embodiments, the machine learning and/or artificial intelligence models may be configured to utilize a variety of techniques to analyze the signals to determine the mood, emotion, mental state, and/or emotional state of the user. For example, the models may utilize natural language processing to analyze text in the signals (e.g., from responses received from the user) to determine the user's sentiment, mood, and/or emotion (e.g., certain words, phrases, tones, and/or other characteristics relating to language may be indicative of a mood, emotion, etc.). As another example, the models may utilize convolutions, vision transformers, and/or other machine learning techniques to analyze image content of the user, video content of the user, and/or other content of the user to determine whether information in the content corresponds and/or matches with training information indicative of a mood, emotion, emotional state, and/or mental state (e.g., furrowed eyebrows in an image taken of the user may indicate that the user is upset or angry based on the model having been trained based on labeled signals and/or data that indicates that a furrowed eyebrow corresponds to anger or being upset). In certain embodiments, the models may utilize techniques for conducting image segmentation, image classification, content-based image retrieval, and/or object detection on the information in the signals to determine a user's mood, emotion, emotional state, and/or mental state. In certain embodiments, exemplary algorithms that may be utilized by the models of the system 100 to perform any of the steps of the method 700 may include, but are not limited to, classification algorithms, logistic regression algorithms, support vector machine algorithms, Naïve Bayes algorithms, decision trees, ensemble techniques, deep learning algorithms, and/or any other types of algorithms. In certain embodiments, the labeling may be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device. At step 706, the method 700 may include assigning, based on the plurality of signals a score value relating to the mental health of the user. In certain embodiments, the assigning of the score value may be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device.

At step 708, the method 700 may include determining, based on the score value and/or labeling of signals matched with labeling of outputs of the system 100, a deficit or need of the user relating to the mental health of the user. For example, if the score is scaled between 1-10 and the user's mental health has a score value of 3, the system 100 may determine that there is a deficiency or need relating to the mental health because a score of 3 is too far below the optimal score of 10. In certain embodiments, at step 708, the method 700 may include determining the deficit or need of the user relating to the mental health of the user based on the labeling of signals matched with the labeling of outputs. For example, input signals labeled based on an assessment of how user engagement with content is to be interpreted may be compared with outputs of the system 100 (and/or training data utilized to train the machine learning models of the system 100) that have labels correlating, matching, and/or having commonality with the labels of the input signals. If the labels match or have a threshold level of correlation and/or matching, the system 100 may determine that this is a deficiency or need relating to the user's mental health. As an example, if an input signal has interaction data that includes an image of the user that also includes a sad emoticon set by the user for the image and the expressions in the image and/or the sad emoticon match and/or have information in common with training data and/or outputs of the system 100 that are labeled as also being associated with sadness, the user may be determined to have a deficit or need relating to the mental health of the user. In certain embodiments, the determining of the deficit or need may be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device. At step 710, the method 700 may include determining, by utilizing an artificial intelligence model, a prediction relating to content, activities, resources, or a combination thereof, to be presented and/or delivered for interaction with the user to enhance the score value relating to the mental health of the user. The content, activities, and/or resources may include, but are not limited to, surveys, gaming content, mood booster content, videos, training sessions, therapy sessions, virtual meetings with mental health experts, interactive content, virtual reality content, augmented reality content, quizzes, data extraction programs, questionnaires, activities, breathing exercise programs, meditation programs, and/or other types of content, activities, and/or resources. In certain embodiments, the determining of the predictions may be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device.

At step 712, the method 700 may include providing access to the content, activities, resources, or a combination thereof, to a device of the user. In certain embodiments, the providing of the access may be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device. At step 714, the method 700 may include dynamically adjusting the score value relating to the mental health of the user in real-time as the user interacts with the content, activities, resources, or a combination thereof. For example, signals provided by the application that include information indicating the mood and/or emotions of the user as the user interacts with the content, activities, resources, or a combination thereof, may be utilized to adjust the score. In certain embodiments, the adjusting of the score value may be performed and/or facilitated by utilizing the first user 101, the second user 110 and/or by utilizing the first user device 102, the second user device 111, the server 140, the server 145, the server 150, the server 160, the communications network 135, any component of the system 100, any combination thereof, or by utilizing any other appropriate program, network, system, or device. At step 716, the method 700 may include dynamically adjusting recommendations for content, activities, and/or resources to be presented to the user based on the adjusted score and/or other data received and/or associated with the user. The method 700 may be repeated continuously over time for any number of users to enhance each user's mental health over time in a more expeditious and effective manner than currently possible. In certain embodiments, user interaction data gathered based on a user's interaction with content presented and/or delivered to the user by the system 100 may serve as inputs at step 702 of the method 700 at any subsequent iteration of the method 700. For example, such interaction data may include the length of time that the user interacted with content, whether the user shared the content with others, whether the user scrolled in the application to access additional content, how the user interacted with the content (e.g., did the user like the content, dislike the content, only experience a portion of the content, shared the content with others, etc.), along with any other data generated and/or obtained by the application and/or system 100 based on the user's interaction with the content or lack thereof. By incorporating such data at step 702, the predictions generated by the system 100 relating to recommendations for future content to be presented and/or served to a user will be enhanced significantly over time. Additionally, the interaction data may be utilized by the system 100 to train the models used by the system 100 to generate recommendations on a separate occasion for that same user or potentially other users that may have some correlation to the user or to the user's behavior. Notably, the method 700 may further incorporate any of the features and functionality described for the system 100, any other method disclosed herein, or as otherwise described herein.

The systems and methods disclosed herein may include still further functionality and features. For example, the operative functions of the system 100 and method may be configured to execute on a special-purpose processor specifically configured to carry out the operations provided by the system 100 and method. Notably, the operative features and functionality provided by the system 100 and method may increase the efficiency of computing devices that are being utilized to facilitate the functionality provided by the system 100 and the various methods discloses herein. For example, by training the system 100 over time based on data and/or other information provided and/or generated in the system 100, a reduced amount of computer operations may need to be performed by the devices in the system 100 using the processors and memories of the system 100 than compared to traditional methodologies. In such a context, less processing power needs to be utilized because the processors and memories do not need to be dedicated for processing. As a result, there are substantial savings in the usage of computer resources by utilizing the software, techniques, and algorithms provided in the present disclosure. In certain embodiments, various operative functionality of the system 100 may be configured to execute on one or more graphics processors and/or application specific integrated processors.

Notably, in certain embodiments, various functions and features of the system 100 and methods may operate without any human intervention and may be conducted entirely by computing devices. In certain embodiments, for example, numerous computing devices may interact with devices of the system 100 to provide the functionality supported by the system 100. Additionally, in certain embodiments, the computing devices of the system 100 may operate continuously and without human intervention to reduce the possibility of errors being introduced into the system 100. In certain embodiments, the system 100 and methods may also provide effective computing resource management by utilizing the features and functions described in the present disclosure. For example, in certain embodiments, devices in the system 100 may transmit signals indicating that only a specific quantity of computer processor resources (e.g. processor clock cycles, processor speed, etc.) may be devoted to training the artificial intelligence model(s), generating predictions relating to mental health improvement or regression, generating predictions relating to optimal or ideal interactions to present to a user, and/or performing any other operation conducted by the system 100, or any combination thereof. For example, the signal may indicate a number of processor cycles of a processor may be utilized to update and/or train an artificial intelligence model, and/or specify a selected amount of processing power that may be dedicated to generating or any of the operations performed by the system 100. In certain embodiments, a signal indicating the specific amount of computer processor resources or computer memory resources to be utilized for performing an operation of the system 100 may be transmitted from the first and/or second user devices 102, 111 to the various components of the system 100.

In certain embodiments, any device in the system 100 may transmit a signal to a memory device to cause the memory device to only dedicate a selected amount of memory resources to the various operations of the system 100. In certain embodiments, the system 100 and methods may also include transmitting signals to processors and memories to only perform the operative functions of the system 100 and methods at time periods when usage of processing resources and/or memory resources in the system 100 is at a selected value. In certain embodiments, the system 100 and methods may include transmitting signals to the memory devices utilized in the system 100, which indicate which specific sections of the memory should be utilized to store any of the data utilized or generated by the system 100. Notably, the signals transmitted to the processors and memories may be utilized to optimize the usage of computing resources while executing the operations conducted by the system 100. As a result, such functionality provides substantial operational efficiencies and improvements over existing technologies.

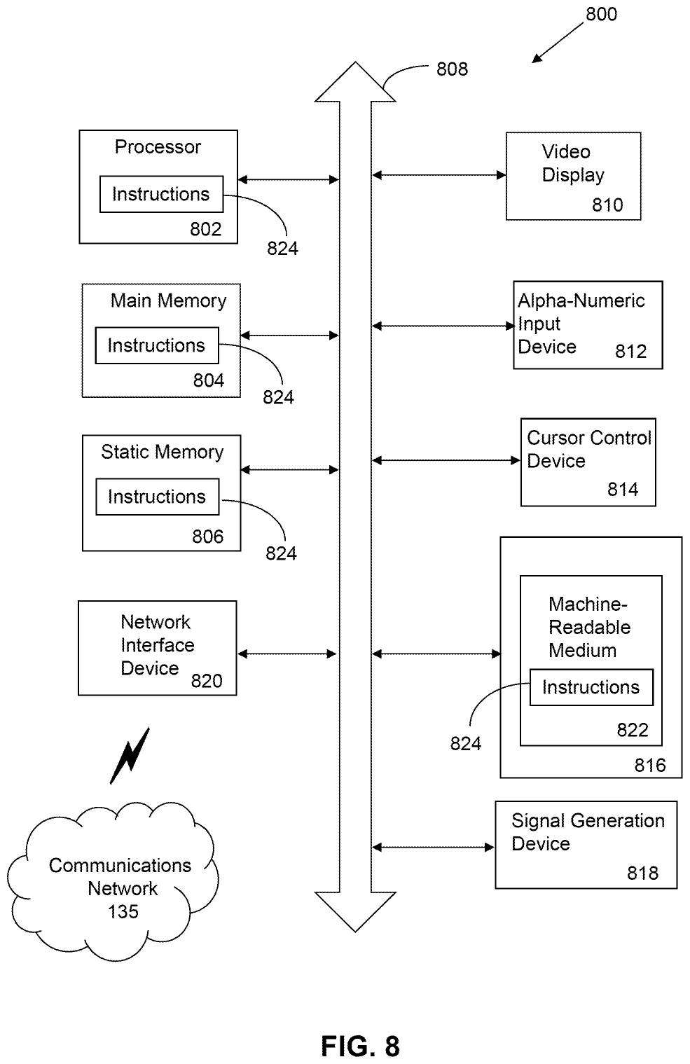

Referring now also to FIG. 8, at least a portion of the methodologies and techniques described with respect to the exemplary embodiments of the system 100 can incorporate a machine, such as, but not limited to, computer system 800, or other computing device within which a set of instructions, when executed, may cause the machine to perform any one or more of the methodologies or functions discussed above. The machine may be configured to facilitate various operations conducted by the system 100. For example, the machine may be configured to, but is not limited to, assist the system 100 by providing processing power to assist with processing loads experienced in the system 100, by providing storage capacity for storing instructions or data traversing the system 100, or by assisting with any other operations conducted by or within the system 100. As another example, the computer system 800 may assist with determining interactions to conduct with a user, determining types of content to suggest to a user, analyze user interactions conducted within an application of the system, assessing the mental health of a user, assessing the mental health improvement of the user, evaluating the mental health of a user, evaluating the mental health improvement of the user, adapting artificial intelligence models supporting the functionality of the system 100 as inputs and/or data change over time, and/or performing any other operations of the system 100.

In some embodiments, the machine may operate as a standalone device. In some embodiments, the machine may be connected (e.g., using communications network 135, another network, or a combination thereof) to and assist with operations performed by other machines and systems, such as, but not limited to, the first user device 102, the second user device 111, the server 140, the server 145, the server 150, the database 155, the server 160, any other system, program, and/or device, or any combination thereof. The machine may be connected with any component in the system 100. In a networked deployment, the machine may operate in the capacity of a server or a client user machine in a server-client user network environment, or as a peer machine in a peer-to-peer (or distributed) network environment. The machine may comprise a server computer, a client user computer, a personal computer (PC), a tablet PC, a laptop computer, a desktop computer, a control system, a network router, switch or bridge, or any machine capable of executing a set of instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies discussed herein.

The computer system 800 may include a processor 802 (e.g., a central processing unit (CPU), a graphics processing unit (GPU, or both), a main memory 804 and a static memory 806, which communicate with each other via a bus 808. The computer system 800 may further include a video display unit 810, which may be, but is not limited to, a liquid crystal display (LCD), a flat panel, a solid-state display, or a cathode ray tube (CRT). The computer system 800 may include an input device 812, such as, but not limited to, a keyboard, a cursor control device 814, such as, but not limited to, a mouse, a disk drive unit 816, a signal generation device 818, such as, but not limited to, a speaker or remote control, and a network interface device 820.

The disk drive unit 816 may include a machine-readable medium 822 on which is stored one or more sets of instructions 824, such as, but not limited to, software embodying any one or more of the methodologies or functions described herein, including those methods illustrated above. The instructions 824 may also reside, completely or at least partially, within the main memory 804, the static memory 806, or within the processor 802, or a combination thereof, during execution thereof by the computer system 800. The main memory 804 and the processor 802 also may constitute machine-readable media.

Dedicated hardware implementations including, but not limited to, application specific integrated circuits, programmable logic arrays and other hardware devices can likewise be constructed to implement the methods described herein. Applications that may include the apparatus and systems of various embodiments broadly include a variety of electronic and computer systems. Some embodiments implement functions in two or more specific interconnected hardware modules or devices with related control and data signals communicated between and through the modules, or as portions of an application-specific integrated circuit. Thus, the example system is applicable to software, firmware, and hardware implementations.

In accordance with various embodiments of the present disclosure, the methods described herein are intended for operation as software programs running on a computer processor. Furthermore, software implementations can include, but not limited to, distributed processing or component/object distributed processing, parallel processing, or virtual machine processing can also be constructed to implement the methods described herein.

The present disclosure contemplates a machine-readable medium 822 containing instructions 824 so that a device connected to the communications network 135, another network, or a combination thereof, can send or receive voice, video or data, and communicate over the communications network 135, another network, or a combination thereof, using the instructions. The instructions 824 may further be transmitted or received over the communications network 135, another network, or a combination thereof, via the network interface device 820.

While the machine-readable medium 822 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that causes the machine to perform any one or more of the methodologies of the present disclosure.

The terms “machine-readable medium,” “machine-readable device,” or “computer-readable device” shall accordingly be taken to include, but not be limited to: memory devices, solid-state memories such as a memory card or other package that houses one or more read-only (non-volatile) memories, random access memories, or other re-writable (volatile) memories; magneto-optical or optical medium such as a disk or tape; or other self-contained information archive or set of archives is considered a distribution medium equivalent to a tangible storage medium. The “machine-readable medium,” “machine-readable device,” or “computer-readable device” may be non-transitory, and, in certain embodiments, may not include a wave or signal per se. Accordingly, the disclosure is considered to include any one or more of a machine-readable medium or a distribution medium, as listed herein and including art-recognized equivalents and successor media, in which the software implementations herein are stored.

The illustrations of arrangements described herein are intended to provide a general understanding of the structure of various embodiments, and they are not intended to serve as a complete description of all the elements and features of apparatus and systems that might make use of the structures described herein. Other arrangements may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

Thus, although specific arrangements have been illustrated and described herein, it should be appreciated that any arrangement calculated to achieve the same purpose may be substituted for the specific arrangement shown. This disclosure is intended to cover any and all adaptations or variations of various embodiments and arrangements of the invention. Combinations of the above arrangements, and other arrangements not specifically described herein, will be apparent to those of skill in the art upon reviewing the above description. Therefore, it is intended that the disclosure is not limited to the particular arrangement(s) disclosed as the best mode contemplated for carrying out this invention, but that the invention will include all embodiments and arrangements falling within the scope of the appended claims.

The foregoing is provided for purposes of illustrating, explaining, and describing embodiments of this invention. Modifications and adaptations to these embodiments will be apparent to those skilled in the art and may be made without departing from the scope or spirit of this invention. Upon reviewing the aforementioned embodiments, it would be evident to an artisan with ordinary skill in the art that said embodiments can be modified, reduced, or enhanced without departing from the scope and spirit of the claims described below.

We claim:

1. A system, comprising:
a memory that stores instructions; and
a processor that executes the instructions to perform operations, the operations comprising:
capturing, via an application, a plurality of signals associated with a mood, a mental state, or a combination thereof, associated with a user, interaction data associated with the user, or a combination thereof;
labeling the plurality of signals based on an assessment of how engagement with content is to be interpreted;
assigning, based on the plurality of signals, a score value relating to a mental health of the user;
determining, based on the score value, the plurality of signals, or a combination thereof, a deficit or need of the user relating to the mental health of the user;
determining, by utilizing an artificial intelligence model, a prediction relating to content, activities, resources, or a combination thereof, to be presented or delivered for interaction with the user to enhance the score value relating to the mental health of the user;
providing access to the content, activities, resources, or a combination thereof, to a device of the user; and
dynamically adjusting the score value relating to the mental health of the user in real-time as the user interacts with the content, activities, resources, or a combination thereof.

2. The system of claim 1, wherein the operations further comprise dynamically adjusting recommendations for content, activities, resources, or a combination thereof, in real-time as the score value is adjusted, as the user interacts with the content, activities, or resources, as circumstances relating to the user change, or a combination thereof.

3. The system of claim 1, wherein the content, activities, or resources comprise one or more of a coaching session, a crisis resource, a community resource, a care activity, a therapy session, video content, gaming content, audio content, virtual reality content, augmented reality content, medical content, medical therapy sessions, surveys, mental health content, training content, or a combination thereof.

4. The system of claim 1, wherein the score value is comprised of score values relating to a plurality of variables utilized in determining the mental health of the user, and wherein the plurality of variables comprises one or more of a relationship variable, a physical variable, a mind variable, a positivity variable, an engagement variable, a mood tracker variable, a mental state variable, or a combination thereof.

5. The system of claim 1, wherein the operations further comprise capturing the plurality of signals from the device of the user as the user interacts with the application.

6. The system of claim 1, wherein the operations further comprise generating the content, activities, resources, or a combination thereof, such that the content, resources, or a combination thereof, are tailored to improving the mental health of the user.

7. The system of claim 1, wherein the operations further comprise capturing the plurality of signals on a continuous basis or while the user is utilizing the application.

8. The system of claim 1, wherein the operations further comprise training the artificial intelligence model based on information associated with the user and other users, information known to be associated with mental health, patterns associated with generating mental health diagnoses, information associated types of content to enhance mental health, or a combination thereof.

9. The system of claim 1, wherein the operations further comprise adjusting a confidence score for the prediction based on whether the content, the activities, or the resources, affected the mental health of the user in a manner as predicted.

10. The system of claim 1, wherein the operations further comprise visually rendering the score value in a graph on a user interface of the device of the user, and wherein the operations further comprise visually rendering sub-scores of variables utilized in determining the score in the graph.

11. The system of claim 1, wherein the operations further comprise determining which content, activities, or resources generated the highest improvement to the score value relating to the mental health of the user.

12. The system of claim 11, wherein the operations further comprise providing a higher weight to the content, the activities, or the resources that generated the highest improvement to the score value in comparison to other content, other activities, or other resources.

13. A method, comprising
capturing, via an application and by utilizing instructions from a memory that are executed by a processor, a plurality of signals associated with a mood, a mental state, or a combination thereof, associated with a user, interaction data associated with the user, or a combination thereof;
labeling the plurality of signals based on an assessment of how engagement with content is to be interpreted;
assigning, based on the plurality of signals, a score value relating to a mental health of the user;
determining, based on the score value, the plurality of signals, or a combination thereof a deficit or need of the user relating to the mental health of the user;
determining, by utilizing an artificial intelligence model, a prediction relating to content, activities, resources, or a combination thereof, to be presented or delivered for interaction with the user to enhance the score value relating to the mental health of the user;
providing access to the content, activities, resources, or a combination thereof, to a device of the user; and
dynamically adjusting the score value relating to the mental health of the user in real-time as the user interacts with the content, activities, resources, or a combination thereof.

14. The method of claim 13, further comprising training the artificial intelligence model based on interactions with the content, the activities, the resources, or a combination thereof.

15. The method of claim 13, further comprising providing access to the content, activities, resources, or a combination thereof, to the device of the user via a user interface of the application.

16. The method of claim 13, further comprising recommending content, activities, resources, or a combination thereof, to the user based on the user having a threshold correlation with characteristics of another user.

17. The method of claim 13, further comprising updating the adjusting a predictive capability of the artificial intelligence model based on interactions conducted by the user with respect to the content, activities, resources, or a combination thereof.

18. The method of claim 14, further comprising capturing sensor data including biometric data, visual content of the user, audio content associated with the user, virtual reality content associated with the user, augmented reality content associated with the user, or a combination thereof.

19. The method of claim 14, further comprising visually rendering adjustments to the score value to a user interface of the device of the user in real-time to advise the user to the adjustments to the score value based on interactions with the content, the activities, the resources, or a combination thereof.

20. A computer-readable device comprising instructions, which, when loaded and executed by a processor, cause the processor to perform operations, the operations comprising:
capturing, via an application, a plurality of signals associated with a mood, a mental state, or a combination thereof, associated with a user, interaction data associated with the user, or a combination thereof;
labeling the plurality of signals based on an assessment of how engagement with content is to be interpreted;
assigning, based on the plurality of signals, a score value relating to a mental health of the user;
determining, based on the score value, the plurality of signals, or a combination thereof, a deficit or need of the user relating to the mental health of the user;
determining, by utilizing an artificial intelligence model, a prediction relating to content, activities, resources, or a combination thereof, to be presented or delivered for interaction with the user to enhance the score value relating to the mental health of the user;
providing access to the content, activities, resources, or a combination thereof, to a device of the user; and
dynamically adjusting the score value relating to the mental health of the user in real-time as the user interacts with the content, activities, resources, or a combination thereof.
